(19) United States
(12) Patent Application Publication
(10) Pub. No.: US 2007/0264200 A1
(43) Pub. Date: Nov. 15, 2007

(54) Mouse MRI for drug screening

(76) Inventors: Scott Small, Millerton, NY (US); Herman Moreno, New York, NY (US)

Correspondence Address: COOPER & DUNHAM, LLP, 1185 AVENUE OF THE AMERICAS, NEW YORK, NY, 10036, US

(21) Appl. No.: 11/598,938

(22) Filed: Nov. 13, 2006

Related U.S. Application Data
(60) Provisional application No. 60/736,630, filed on Nov. 14, 2005.

(51) Int. Cl. A61B 5/055

(52) U.S. Cl. 424/9.36

(57) ABSTRACT

This invention provides a method for determining the amount of blood in a volume of cerebral tissue (cerebral blood volume) in a mammalian subject comprising (a) acquiring a first magnetic resonance image of the volume of tissue in vivo; (b) administering intraperitoneally to the subject a gadolinium-containing contrast agent in an amount greater than about 1 mg per kg body weight and less than about 20 mg per kg body weight; (c) acquiring a second magnetic resonance image of the volume of tissue in vivo, which second image is acquired at least about 15 minutes after, but not more than about 2 hours after, administering the contrast agent; and (d) determining the amount of cerebral blood volume based on the first and second images.

This application claims the benefit of U.S. Provisional Application No. 60/736,630, filed Nov. 14, 2005, the contents of which are incorporated herein by reference into the subject application.

This invention was made with support under United States Government Grant Nos. AG07232, AG08702, and NS43469 from the National Institutes of Health. Accordingly, the United States Government has certain rights in the subject invention.

Throughout this application, certain publications are referenced. Full citations for these publications, as well as additional related references, may be found immediately preceding the claims. The disclosures of these publications are hereby incorporated by reference into this application in order to more fully describe the state of the art as of the date of the invention described and claimed herein.

### BACKGROUND OF THE INVENTION

Since the early studies performed by Kety and Schmidt (1) measurements to detect changes in regional energy metabolism have been extensively used, including functional brain imaging. In this context, regional energy metabolism is the local rate at which cells produce/consume ATP, which requires the consumption of oxygen and glucose (in neurons) from the blood. Visualizing ATP production directly is challenging, but imaging techniques have been developed that can visualize correlates of oxygen and glucose consumption caused by the coupling between energy demand and supply which leads to changes in cerebral blood flow and volume when the local energy metabolism changes. Because of this hemodynamic coupling, regional oxygen consumption is well correlated with regional deoxyhemoglobin content (2-4), regional cerebral blood flow (rCBF) (5,6) and regional cerebral blood volume (rCBV) (7,8).

Positron Emission Tomography (PET) can directly estimate the regional rates of both glucose and oxygen consumption through the use of radiolabeled glucose analogs and ¹⁵O water and O2. These techniques work well in humans and larger primates but do not have an adequate spatial resolution to use in mice.

Spatial resolution is particularly important in this case because diseases often begin by targeting small subregions within a brain structure. In contrast, MRI-based techniques have excellent spatial resolution and, relying on the hemodynamic coupling between oxygen consumption—rCBV and rCBF, have been able to visualize variations of energy metabolism at the submillimeter scale in mice.

Neurological diseases typically affect the basal metabolic rate of oxygen consumption in specific brain subregions, which can then be used to map the anatomical sites of dysfunction. Indeed, the basal changes of energy metabolism associated with diseases have been detected relying on all metabolic correlates—glucose uptake, CBF, CBV, and deoxyhemoglobin content. Nevertheless, CBV measurements with MRI have proven to be ideally suited to map dysfunction with regional precision in the mouse brain (9). Compared to glucose uptake and CBF, CBV measurements can be generated with significantly higher spatial resolution (10); and, compared to measures of basal deoxyhemoglobin, CBV can be measured with greater precision (11).

A number of approaches have been developed to generate high-resolution CBV maps of the mouse and rat brain (12-13). In these techniques, CBV is calculated from the signal change caused after the intravenous (IV) injection of an iron-containing or gadolinium based contrast agents (12-14). These approaches have a number of limitations for mapping CBV longitudinally over time. First, unlike larger animals, gaining venous access in a mouse is not trivial. Typically, a cut-down surgical procedure is used to access a large vein, a traumatic procedure associated with frequent morbidity and occasional mortality. Alternatively one of the two tail veins can be used, but these veins cannot be probed multiple times. A second limitation is the use of iron-containing contrast agents, which may lead to organ toxicity when used at high-doses or repeatedly (15), limiting the number of times a CBV map can be generated over time, which is required for mapping the spatiotemporal pattern of disease onset and progression.

### SUMMARY OF THE INVENTION

This invention provides a method for determining the amount of blood in a volume of cerebral tissue (cerebral blood volume) in a mammalian subject comprising (a) acquiring a first magnetic resonance image of the volume of tissue in vivo; (b) administering intraperitoneally to the subject a gadolinium-containing contrast agent in an amount greater than about 1 mg per kg body weight and less than about 20 mg per kg body weight; (c) acquiring a second magnetic resonance image of the volume of tissue in vivo, which second image is acquired at least about 15 minutes after, but not more than about 2 hours after, administering the contrast agent; and (d) determining the amount of cerebral blood volume based on the first and second images.

This invention also provides a method for determining the change in the amount of blood in a volume of cerebral tissue (cerebral blood volume) in a mammalian subject over a predefined period of time, comprising determining the cerebral blood volume at a plurality of time points during the predefined period of time and comparing the cerebral blood volumes so determined, so as to determine the change in the cerebral blood volume over the predefined period of time, wherein at each time point, determining the cerebral blood volume is performed according to the above-described method, with the proviso that at each time point other than the final time point in the predefined period of time, a saline solution is intraperitoneally administered to the subject following either step (c) or step (d).

This invention also provides a method for determining, in an animal model for a human neurological disease correlative with a decrease in cerebral blood volume, whether an agent inhibits the decrease, comprising (a) administering the agent to the subject; (b) determining the cerebral blood volume of a suitable volume of the subject's cerebral tissue at a plurality of time points during a predefined period of time, wherein step (a) is performed before and/or during the predefined period; and wherein determining the cerebral blood volume is performed according to the above-described method, with the proviso that at each time point other than the final time point in the predefined period of time, a saline solution is intraperitoneally administered to the subject following either step (c) or step (d) the above-described method; (c) comparing the cerebral blood volumes so determined, so as to determine the change in the cerebral blood volume over the predefined period of time; and (d) comparing the change determined in step (c) with the change determined in a subject to which the agent was not administered, thereby determining whether the agent inhibits the decrease in cerebral blood volume correlative with the disease.

### BRIEF DESCRIPTION OF THE FIGURES

FIG. 1A-C

Characteristics of IP gadodiamide generated ΔR2 maps.

A. Dose response curves of ΔR2 in sec-1 (Delta R2 [1/sec]) as a function of IP gadodiamide dose, in different brain regions (means values). Post contrast images were obtained at 45 minutes after injection.

B. Time course of change in relaxation rate ΔR2 (Delta R2 [1/sec]) induced by 10 mmol/kg IP gadodiamide in different brain areas (mean values).

C. Calculated contrast to noise ration (CNR) in relation to IP gadodiamide dose, calculation was done as described in methods. Brain regions for A&B were defined as: Cortex (parietal cortex), Striatum (caudate and putamen), Thalamus (thalamus proper-medial, and lateral nuclei), Hippo (Subicullum, CA Subfields and Dentate Gyrus).

FIG. 2A-C

Comparison of CBV maps obtained with different methods.

A. Shown are the mean values of ΔR2 in sec-1 (Delta R2 [1/sec]) in different ROIs, bars denote SDM (methods included: IV Feri=18 mg Fe/kg feridex, IV Gado=0.2 mmol/kg gadodiamide, IP High Gado=10 mmol/kg gadodiamide and IP Gado=5 mmol/kg gadodiamide). Post contrast images for IV experiments were obtained at 3.75 minutes

B. normalized ΔR2 maps as: (mean ΔR2 (roi)/maximum {4 pixels} ΔR2 at the posterior cerebral vessel) (see text for details, also illustrated in FIG. 3).

C. Regions of interest (ROI) in a mouse brain T2 (FSE) MRI.

FIG. 3A-F

Generation of CBV maps.

A. T2 weighted image, axial slice at the level of the mid body of hippocampal formation (PRE)

B. T2 weighted image after 45 minutes of 10 mmol/kp IP gadodiamide (POST)

C. ΔR2 map generated with A&B, as described in the text.

D. T2* weighted mage at the same location as (A)

E. Post contrast T2* weighted image utilizing same conditions as in (B)

F. ΔR2* map generated with D&E.

FIG. 4A-C

Measuring CBV longitudinally with MRI. Sown area three ΔR2 maps from the same mouse during three different imaging sessions using the same protocol each time, with a one-week interval between images A-C. The values of the ΔR2 are very similar (mean ΔR2 whoe brain, A=6.1 B=6.2)

### DETAILED DESCRIPTION OF THE INVENTION

Definitions

As used in this application, except as otherwise expressly provided herein, each of the following terms shall have the meaning set forth below.

As used herein, “administering” an agent can be effected or performed using any of the various methods and delivery systems known to those skilled in the art. The administering can be performed, for example, intravenously, intraperitoneally, via cerebrospinal fluid, orally, nasally, via implant, transmucosally, transdermally, intramuscularly, and subcutaneously.

As used herein, “agent” shall mean any chemical entity, including, without limitation, a protein, an antibody, a nucleic acid, a small molecule, and any combination thereof.

As used herein, “cerebral blood volume” shall mean (i) the volume of blood present in a volume of cerebral tissue, or (ii) a quantitative value (e.g. 1 μm³) correlative either with the volume of blood present in a volume of cerebral tissue and/or with the metabolic activity in that volume of cerebral tissue.

As used herein, “gadolinium-containing contrast agent” shall mean, where used with respect to brain imaging, any gadolinium-containing substance administrable to a subject which results in an intravascular enhancement. Examples of gadolinium-containing contrast agents are gadolinium, gadolinium pentate and gadodiamide.

As used herein, “subject” shall mean any animal, such as a human, non-human primate, mouse, rat, guinea pig or rabbit.

### EMBODIMENTS OF THE INVENTION

This invention provides a method for determining the amount of blood in a volume of cerebral tissue (cerebral blood volume) in a mammalian subject comprising (a) acquiring a first magnetic resonance image of the volume of tissue in vivo; (b) administering intraperitoneally to the subject a gadolinium-containing contrast agent in an amount greater than about 1 mg per kg body weight and less than about 20 mg per kg body weight; (c) acquiring a second magnetic resonance image of the volume of tissue in vivo, which second image is acquired at least about 15 minutes after, but not more than about 2 hours after, administering the contrast agent; and (d) determining the amount of cerebral blood volume based on the first and second images.

In one embodiment, the subject is a mouse. In another embodiment, the gadolinium-containing contrast agent is selected from the group consisting of gadolinium, gadolinium pentate, and gadodiamide. In the preferred embodiment, the gadolinium-containing contrast agent is gadolinium pentate. In a further embodiment, the amount of the gadolinium-containing contrast agent is administered in an amount of about 10 mg per kg body weight. In another embodiment, the second magnetic resonance image is acquired about 45 minutes after administering the gadolinium-containing contrast agent. This invention also provides the above-described method further comprising the step of intraperitoneally administering a saline solution (e.g. Ringer's solution) to the subject, which administering follows either step (c) or step (d).

In one embodiment, the subject is a mouse and at least about 4 ml of saline solution is administered. In another embodiment, the subject is a mouse and about 5 ml of saline solution is administered. In yet another embodiment, the subject is an animal model for a human neurological disease.

In one embodiment, the animal model is a transgenic mouse model for Alzheimer's disease whose cells express a mutant Ameloid Precursor Protein (APP), thereby causing an increase in Aβ peptide in the mouse's brain.

This invention provides a method for determining the change in the amount of blood in a volume of cerebral tissue (cerebral blood volume) in a mammalian subject over a predefined period of time, comprising determining the cerebral blood volume at a plurality of time points during the predefined period of time and comparing the cerebral blood volumes so determined, so as to determine the change in the cerebral blood volume over the predefined period of time, wherein at each time point, determining the cerebral blood volume is performed according to the above-described method, with the proviso that at each time point other than the final time point in the predefined period of time, a saline solution is intraperitoneally administered to the subject following either step (c) or step (d).

In one embodiment, the predefined period of time is one month or longer. In another embodiment, the predefined period of time is six month or longer. In yet another embodiment, the predefined period of time is one year or longer. In a further embodiment, the predefined period of time is two years or longer.

In one embodiment, the plurality of time points during the predefined period of time number 3 or more. In another embodiment, the plurality of time points during the predefined period of time number 5 or more.

In yet another embodiment, the plurality of time points during the predefined period of time number 10 or more. In a further embodiment, the plurality of time points during the predefined period of time number 20 or more.

In one embodiment, the subject is a mouse.

In another embodiment, the gadolinium-containing contrast agent is selected from the group consisting of gadolinium, gadolinium pentate, and gadodiamide. In yet another embodiment, the gadolinium-containing contrast agent is gadolinium pentate. In a further embodiment, the amount of the gadolinium-containing contrast agent is administered in an amount of about 10 mg per kg body weight.

In one embodiment, the second magnetic resonance image is acquired about 45 minutes after administering the gadolinium-containing contrast agent. In another embodiment, the subject is a mouse and at least about 4 ml of saline solution is administered. In yet another embodiment, the subject is a mouse and about 5 ml of saline solution is administered.

In a preferred embodiment, the subject is an animal model for a human neurological disease. In one embodiment, the animal model is a transgenic mouse model for Alzheimer's disease whose cells express a mutant Ameloid Precursor Protein (APP), thereby causing an increase in Aβ peptide in the mouse's brain. In another embodiment, a decrease in cerebral blood volume over the predefined period of time correlates with the progression of disease in the subject. In yet another embodiment, before and/or during the predefined period of time, the subject has administered to it an agent which is being tested for its ability to inhibit the decrease in cerebral blood volume over the predefined period of time.

This invention also provides a method for determining, in an animal model for a human neurological disease correlative with a decrease in cerebral blood volume, whether an agent inhibits the decrease, comprising (a) administering the agent to the subject; (b) determining the cerebral blood volume of a suitable volume of the subject's cerebral tissue at a plurality of time points during a predefined period of time, wherein step (a) is performed before and/or during the predefined period; and wherein determining the cerebral blood volume is performed according to the above-described method with the proviso that at each time point other than the final time point in the predefined period of time, a saline solution is intraperitoneally administered to the subject following either step (c) or step (d) of the above-described method; (c) comparing the cerebral blood volumes so determined, so as to determine the change in the cerebral blood volume over the predefined period of time; and (d) comparing the change determined in step (c) with the change determined in a subject to which the agent was not administered, thereby determining whether the agent inhibits the decrease in cerebral blood volume correlative with the disease.

In one embodiment, the predefined period of time is one month or longer. In another embodiment, the predefined period of time is six month or longer. In yet another embodiment, the predefined period of time is one year or longer. In a further embodiment, the predefined period of time is two years or longer.

In one embodiment, the plurality of time points during the predefined period of time number 3 or more. In another embodiment, the plurality of time points during the predefined period of time number 5 or more. In yet another embodiment, the plurality of time points during the predefined period of time number 10 or more. In a further embodiment, the plurality of time points during the predefined period of time number 20 or more.

In one embodiment, the subject is a mouse. In another embodiment, the gadolinium-containing contrast agent is selected from the group consisting of gadolinium, gadolinium pentate, and gadodiamide. In the preferred embodiment, the gadolinium-containing contrast agent is gadolinium pentate.

In one embodiment, the amount of the gadolinium-containing contrast agent is administered in an amount of about 10 mg per kg body weight. In another embodiment, the second magnetic resonance image is acquired about 45 minutes after administering the gadolinium-containing contrast agent.

In one embodiment, the subject is a mouse and at least about 4 ml of saline solution is administered. In another embodiment, the subject is a mouse and about 5 ml of saline solution is administered.

In a preferred embodiment, the subject is an animal model for a human neurological disease. In one embodiment, the animal model is a transgenic mouse model for Alzheimer's disease whose cells express a mutant Ameloid Precursor Protein (APP), thereby causing an increase in Aβ peptide in the mouse's brain. In another embodiment, the agent is an organic compound. In yet another embodiment, the agent is administered orally or intravenously.

This invention is illustrated in the Experimental Details section which follows. This section is set forth to aid in an understanding of the invention but is not intended to, and should not be construed to limit in any way the invention as set forth in the claims which follow thereafter.

### EXPERIMENTAL DETAILS

Synopsis

The ability to conduct repeated measurements of regional cerebral blood volume (rCBV) provides an excellent method of mapping the onset and progression of brain dysfunction in transgenic mouse models of disease. Currently, MRI techniques are the best way to carry out such measurements in mice. Unfortunately, most MRI methods that estimate rCBV rely on intravenous (IV) injections of contrast agents, which are difficult to repeat in mice, making multiple separate measurements problematic. Here, this limitation is addressed by introducing an approach for mapping rCBV that relies on intraperitoneal (IP) rather than IV injections of gadodiamide. After optimization of the procedure, comparisons of CBV maps generated with IP or with IV injections show that CBV maps generated with IP gadodiamide, can be acquired repeatedly and reliably, over a three week period without harm to the mouse.

Introduction

Since the early studies performed by Kety and Schmidt (1) measurements to detect changes in regional energy metabolism have been extensively used, including functional brain imaging. In this context, regional energy metabolism is the local rate at which cells produce/consume ATP, which requires the consumption of oxygen and glucose (in neurons) from the blood. Visualizing ATP production directly is challenging, but imaging techniques have been developed that can visualize correlates of oxygen and glucose consumption caused by the coupling between energy demand and supply which leads to changes in cerebral blood flow and volume when the local energy metabolism changes. Because of this hemodynamic coupling, regional oxygen consumption is well correlated with regional deoxyhemoglobin content (2-4), regional cerebral blood flow (rCBF) (5,6) and regional cerebral blood volume (rCBV) (7,8).

Positron Emission Tomography (PET) can directly estimate the regional rates of both glucose and oxygen consumption through the use of radiolabeled glucose analogs and ¹⁵O water and O2. These techniques work well in humans and larger primates but do not have an adequate spatial resolution to use in mice.

Spatial resolution is particularly important in this case because diseases often begin by targeting small subregions within a brain structure. In contrast, MRI-based techniques have excellent spatial resolution and, relying on the hemodynamic coupling between oxygen consumption—rCBV and rCBF, have been able to visualize variations of energy metabolism at the submillimeter scale in mice.

Neurological diseases typically affect the basal metabolic rate of oxygen consumption in specific brain subregions, which can then be used to map the anatomical sites of dysfunction. Indeed, the basal changes of energy metabolism associated with diseases have been detected relying on all metabolic correlates—glucose uptake, CBF, CBV, and deoxyhemoglobin content. Nevertheless, CBV measurements with MRI have proven to be ideally suited to map dysfunction with regional precision in the mouse brain (9). Compared to glucose uptake and CBF, CBV measurements can be generated with significantly higher spatial resolution (10); and, compared to measures of basal deoxyhemoglobin, CBV can be measured with greater precision (11).

A number of approaches have been developed to generate high-resolution CBV maps of the mouse and rat brain (12-13). In these techniques, CBV is calculated from the signal change caused after the intravenous (IV) injection of an iron-containing or gadolinium based contrast agents (12-14). These approaches have a number of limitations for mapping CBV longitudinally over time. First, unlike larger animals, gaining venous access in a mouse is not trivial. Typically, a cut-down surgical procedure is used to access a large vein, a traumatic procedure associated with frequent morbidity and occasional mortality. Alternatively one of the two tail veins can be used, but these veins cannot be probed multiple times. A second limitation is the use of iron-containing contrast agents, which may lead to organ toxicity when used at high-doses or repeatedly (15), limiting the number of times a CBV map can be generated over time, which is required for mapping the spatiotemporal pattern of disease onset and progression.

With these limitations in mind, intraperitoneal (IP) injection of gadolinium-based contrast agents was explored. These agents have minimal toxicity even when used repeatedly (16). Given the special characteristics of peritoneal solute transport (reviewed in 17), it is reasonable to expect a rapid exchange between the peritoneal fluid and the plasma; further IP injections are easy to administer and are non-traumatic to mice. After optimizing the MRI pulse sequence, comparisons of the IP injection protocol with IV injections of either gadolinium or iron-based contrast agent showed excellent agreement in estimating rCBV. Repeated testing showed that the IP gadolinium protocol is a robust and reliable technique that can generate CBV maps of the brain multiple times over the life span of the mouse.

Materials and Methods

Theory

Contrast agents produce variations in the MR radio frequency signal intensity due to changes in the longitudinal relaxation time (T1) and/or variation of transverse relaxation time (T₂). Gadolinium based contrast agents affect both T1 and T₂ signals. Recent experiments have developed CBV measurements generated from the steady-sate T1 weighted changes, where contrast agent reaches uniform distribution in the vasculature after intravenous injection (10, 18). Compared to dynamic methods, steady-state measurements can generate CBV maps with much higher spatial resolution, required to image small brain areas such as the subregions of the hippocampal formation (19). The quantification of susceptibility effect produced by a contrast agent is complex and depends on vasculature (size and density), field strength and pulse sequence. Experimental (20) and modeling data (3, 21) show that in spin echo sequences signal arises mostly from small vessels (arterioles, capillaries and venules) and contribution of large vessels to signal changes is negligible, while the gradient echo sequence is more sensitive to larger vessels. Both modeling (3) and empirical measurements (8, 22) indicate an approximately linear relationship between the changes of transverse relaxation rate (AR₂ or AR₂*, R₂=1/T₂) and CBV fraction over the physiologically relevant range. When the contrast agent reaches uniform distribution then CBV maps can be measure from steady state T₂-weighted images.
CBV oo AR₂=ln(Sₚᵣₑ/Sₚₒₛₜ)/TE (1)

Where TE is the effective echo time, Sₚᵣₑ is the T₂ weighted signal before contrast agent administration, and Sₚₒₛₜ is the T₂-weighted signal after contrast agent reaches steady state.

Because in these experiments relative CBV was mapped as AR₂ maps, it can be influenced by body composition of mouse, relative small changes in physiological parameters and mainly by contrast agent type. In order to circumvent these problems, when comparing different methods for generating CBV maps, AR₂ measurements were normalized to the maximum (4 pixels) AR₂ signal in the posterior cerebral vessels (an area of highest contrast agent concentration) as previously done (10).

In this study, the effect of a gadolinium-based compound (gadodiamide) on T₂ weighted signal was used, at a high magnetic field (9,4 T) to generate high spatial resolution CBV maps. Although the exact serum elimination half-life of IP gadodiamide injection in mice is unknown, the experimental data show that it allows for a prolong post contrast acquisition of T₂ weighted signal (Sₚₒₛₜ)

Data Acquisition

Mice and Physiological Monitoring:

Wild type C57BL/6 mice 6-10 months of age (25-30 gr) were used in all experiments. Since variations in physiological processes can influence the MRI signal in the brain, various monitoring devices were used while the mouse was being imaged. Heart rate, respiratory rate and SaO₂ were continuously monitored using pulse oximetry (Model V33304, Sergivet). The probe was attached to the lower abdomen. A rectal temperature was continuously monitored with a thermestor (YSI Precision Thermometer 4000A).

ECG was monitored in a physiogard (SM 785, Brucker) by using subcutaneous silver electrodes from the front limbs and the reference electrode placed at the right posterior limb.

Anesthesia:

Although the heads of the mice were mechanically held in place, head motion had to be minimized with anesthesia. Furthermore anesthesia reduces the fear and anxiety induced by the scanner.

Since MRI measurements of CBV relies on hemodynamic coupling,—the biophysical relation between oxygen metabolism and cerebral blood flow, variations in this coupling would confound the measurements. It is known that the volatile anesthetic, isoflurane does not alter CNS metabolic coupling (23). For this reason, isoflurane was used in most of the experiments except where specified, at a dose (induction phase 3 vol % and maintenance 1.5 vol % at 1 L/min air flow, via a nose cone); other anesthetic combinations such as ketamine/xylazine (K/X) have a similar profile to isoflurane (24). Therefore a comparison between these two modalities was performed; for this, a group of 5 mice of similar age and weight were anesthetized with a dose for induction of Ketamine=75 mg/kg and Xylazine 3.75 mg/kg intraperitoneally, and for maintenance one third of the induction dose was given every 20 minutes during the imaging session.

Contrast Agents and Routes of Administration:

Two contrast agents were used. Gadodiamide (Amersham, Oslo Norway), and feridex (Berlex, N.J. USA), a member of the super-paramagnetic iron oxide family of contrast agents.

For IV experiments the tail vein was dissected and a bolus of contrast agent was injected while the mouse was in the magnet. Gadodiamide was diluted 1:1 in normal saline and Feridex was diluted in 5% dextrose (doses specified below). Animals were sacrificed after IV experiments.

For IP experiments, a 0.6 mm catheter was placed intraperitonealy before imaging. The catheter was secure with 6.0 silk suture materials. Once initial images were acquired (pre contrast), gadolinium was injected IP with variable doses in mmol/Kg. Imaging: All images were performed with a Bruker AVANCE 400WB spectrometer (Bruker NMR, Inc., Billerica, Mass.) with an 89 mm bore 9.4 tesla vertical Brucker magnet (Oxford Instruments Ltd., UK) using a 30 mm-i.d birdcage RF probe and a shielded gradient system (100 G/cm). Three scout scans were first acquired to position the subsequent T₂ weighted images along the standard anatomical orientations in a reproducible manner. Optimal axial images were determined empirically by repositioning the animal and manual shimming. Anatomical landmarks were identified using a mouse brain atlas (25).

T₂-weighted images were obtained with a fast SE (FSE) sequence with TR/TE_{eff}=2000 ms/80 ms, rapid acquisition with relaxation enhancement (RARE) factor=16, FOV=26 mm, acquisition matrix=256×256, 10 slices, with a slice thickness=0.6 mm, slice gap=0.1 mm and NEX=28. The in-plane resolution was 100 μm. Each set of images required 15 minutes; six to eight sets were acquired sequentially. The first set corresponded to the pre-contrast image. As soon as the first set was completed, IP Gadodiamide (GD) was injected, while the mouse was still being imaged. The injection lasted 20-30 seconds. Because feridex, and gadodiamide have a rapid vascular clearance, the IV experiments utilized a NEX=14. All images were acquired utilizing the same dynamic range, so no MR signal resealing was required. Signal to noise ratio (SNR) was calculated as follows: First in a pre contrast baseline T₂ weighted image, mean signal intensity (SI) of an appropriate region of interest (ROI) was measured, then the average standard deviation (stdev) of an identical sized ROI of the surrounding air was defined as noise. Then SNR value was obtained using the following equation:
SNR=mean SI-ROI(brain)/stdev ROI(air). (2)

Typical SNR values range between 15-25. Contrast to noise ratio (CNR) (26) was measured over a range of IP gadolinium injections. Given the effect of gadolinium on T₂ signal, it was calculated as:
CNR=(Mean ROI-SI pre-Mean ROI-SI post)/stdev(air)ROI pre. (3)

Experiments exploring T₂* used 3D gradient echo at the same slice location as 2D FSE experiments. The 3D slab GE acquisition parameters were TR/TE=40 ms/5.5 ms, flip angle=5°, matrix 192×256×16, and NEX=11. The in plane resolution and slice thickness were 85 μm and 600 μm respectively. The corresponding 2D FSE images had the same slice thickness. AR₂* was calculated utilizing equation 1.

### Experiments

### Experiment #1

### Optimizing CBV Maps Generated with IP Gadodiamide

First, the AR₂ dependency on the concentration of GD was examined. 18 mice (three pre group) were imaged with concentrations ranging from 0.5 mmol/kg to 15 mmol/kg of IP. Second, once the optimal non-toxic dose was identified, a time course of AR₂ signal was generated. Four mice were imaged in which the time interval post-contrast was systematically lengthened, from 15 minutes to 120 minutes. The number of imaging sets was increased to 8 in these experiments. Third, CBV maps were generated in 3 mice using both gradient-echo and spin-echo pulse sequences, in order to compare R2* signal changes, which are more sensitive to large vessels and R2 signal changes, which are more sensitive to small vessels and therefore more accurately reflects CBV changes coupled to metabolism.

### Experiment #2

### Comparing CBV Maps Generated with IV and IP Contrast Agents

In order to compare parametric features among different methods for generating CBV maps, 16 mice were subdivided equally into four groups: An IV-feridex group who received 18 mg of Fe/kg IV; an IV-gado group who received 0.2 mmol/kg of GD; a low IP-gado who received 5 mmol/kg of GD; and a high IP-gado who received 10 mmol/kg of GD.

### Experiment #3

### Optimizing Serial Acquisitions of CBV Maps Generated with IP Gadodiamide

The effective elimination half-life and biodistribution of IP GD is not known. Residual levels of GD administered at one time point may interfere with the AR2 generated at future time points. In order to optimize the ability to repeatedly and frequently generate CBV maps over time, the effect of different “flush” volumes administered at the end of an imaging session were tested, on subsequent imaging mice were imaged every week for 3 weeks. Mice were subdivided equally into 4 separate groups, with each group receiving either 0, 2, 4, or 5 cc of normal saline IP slow flush (5 minutes) administered IP after the imaging session.

Results

No significant changes were observed on pulse oxymetry measurements (SaO2), heart rate (HR), respiratory rate (RR), or ECG parameters (ST, T wave morphology, rhythm). Comparisons were made within the groups pre with post contrast agent up to 2 hours after IV (feridex or gadodiamide) or IP GD (10 mmol/kg) (Table 1).

### Experiment #1

### Optimizing CBV Maps Generated with IP Gadodiamide

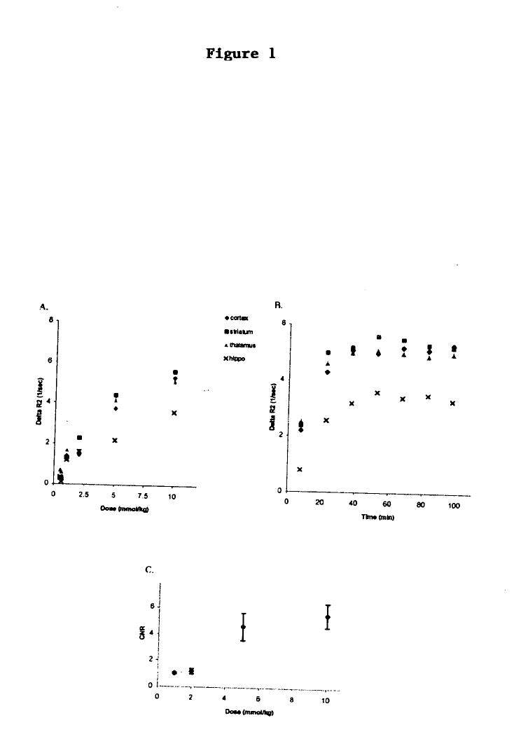

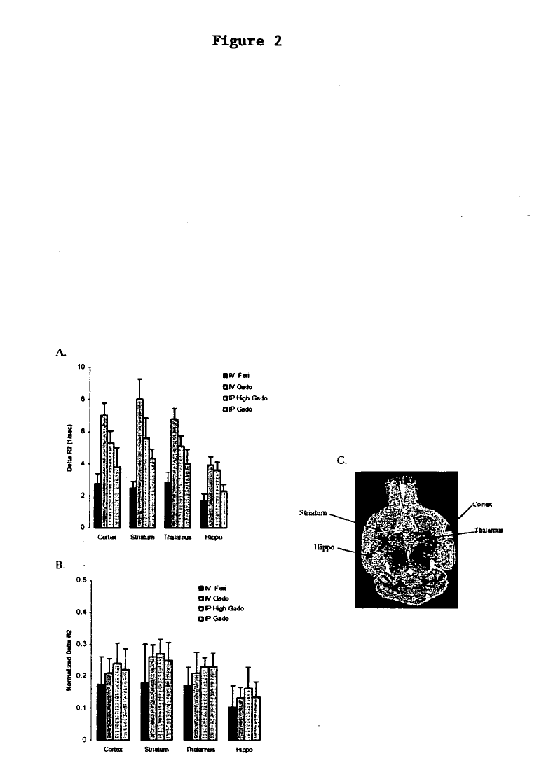

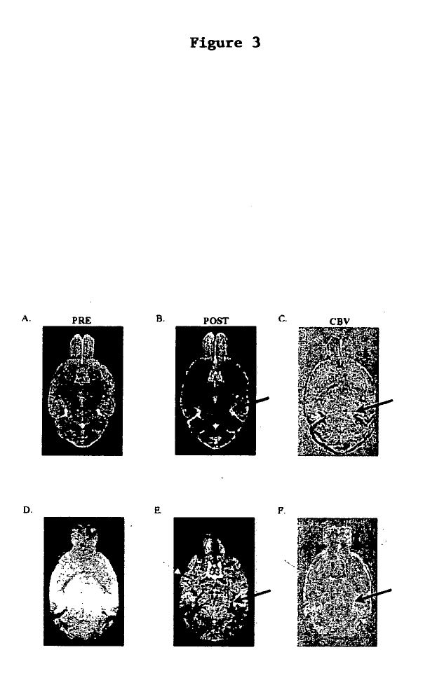

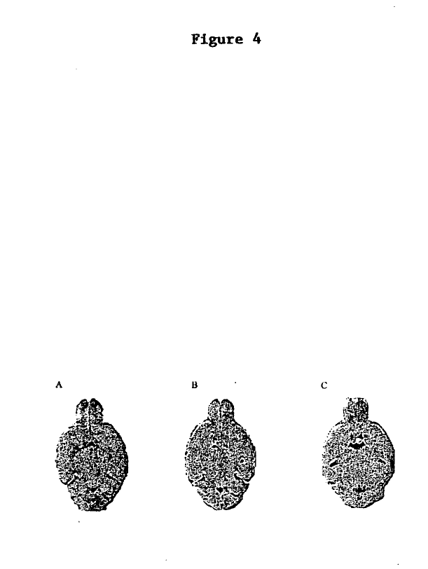

As expected, higher doses of GD resulted in an increase in AR2 and in CNR (FIGS. 1 A&C). However, a 15 mmol/kg dose caused toxicity, manifested as significant changes in vitals signs in two mice (low SaO2 and RR 72/100 and 65/110 respectively), therefore the procedure was aborted. Thus, 10 mmol/kg was identified as the optimum dose of GD, and this dose was used for all further experiment unless otherwise indicated. A time course analysis, where the time interval of post-contrast image was systematically varied, showed that the AR₂ peaked at approximately 37.5 to 53 minutes post-contrast (FIG. 1B). Since the goal is to keep scan time as short as possible, in order to minimize morbidity and mortality, a 45 minutes time point was identified as the optimum post-contrast time interval.

As predicted (3, 12), AR₂* generated with GE pulse sequences were more sensitive to large vessels compared to AR₂ maps generated with FSE pulse sequences (see FIGS. 3 E&F, where small arrows clearly identify cortical vessels). On the other hand, R₂* depends strongly on the experimental conditions and regional field properties, making it difficult to quantify this parameter reproducibly for different regions within a subject and even worse between subjects (27). Therefore, maps generated with AR₂ are preferred.

No significant difference was observed when comparing mice anesthetized with ketamine/xylazine compared to mice anesthetized with isoflurane (t=0.04 p=0.96). Because the levels of isoflurane anesthesia can be more tightly regulated, this form of anesthesia is preferred.

### Experiment #2

### Comparing CBV Maps Generated with IV and IP Contrast Agents

No significant differences (ANOVA: p values>0.05) were observed between the different methods when peak AR₂ values were normalized as described above (FIG. 2B). IV feridex and GD peaked at first acquisition time, while IP doses peaked at an acquisition time equivalent to 37.5 minutes. AR₂ values obtained with IP GD at 10 mmol/kg provides a similar value as those obtained with 18 mg Fe/kg of intravenous MION (12), while IV feridex produced half of that signal (FIG. 2A).

### Experiment #3

### Optimizing Serial Acquisitions of CBV Maps Generated with IP Gadolinium

Low CNR (1.62+/−0.59) was observed in the next imaging session when no flush was administered at the end of an imaging session, with a significant statistical difference when compared to the 2 ml or 4 ml flush group (F=30.0 p=0.0001). Using of 2 ml, 4 or 5 ml flush resulted in high and reliable CNR across the three imaging session, no statistical significant differences were observed between these three groups (F=−1.01 p=0.34). FIG. 4 shows three AR2 maps from the same mouse during three different imaging sessions using the same protocol each time, with a one-week interval between images. The values of the AR2 are very similar (mean AR2 whole brain=5.1, 4.9 and 5.3 respectively).

Discussion

As illustrated in this study, a number of factors need to be considered when using MRI to generate CBV maps in mice. All methods require the use of an intravascular contrast agent, and the choice of which contrast agent to use is the first factor to be considered. Even though one of the main criteria for the generation of steady state CBV maps is a long half-life contrast agent, it does not guarantee that it will produce a sustained intravascular susceptibility perturbation. This was demonstrated empirically with the feridex experiments. A dose of 18 mg/kg of iron from Feridex was used, a dose 32 times higher than that recommended for human use. This dose was selected in order to compare it with published data on MION CBV maps (12). Feridex is a super-paramagnetic iron oxide associated with dextran, which has an elimination half-life in humans of 2.4 hours. The peak signal produced post is about half of that of MION. This may be explained by differences between feridex and MION in the uptake by the reticular endothelial system (28).

The elimination half-life and biodistribution of IV gadodiamide has been carefully measured in humans, mice, and rats (29-30). It has been shown, for instance, that the elimination half-life in humans is approximately 1.5 hours; in rats it is about 20 minutes, and in mice it is 5-6 minutes. This variability is due to differences in glomerular filtration rate. It has also been shown that low levels of residual unchelated gadolinium persist in different tissues for long periods of time after administration, but no measurements in the peritoneum have been reported as of yet (30). In the experiments presented here (FIG. 1B), the time course analysis of IP GD demonstrates that AR₂ remains approximately steady from 37.5 minutes after contrast administration up to 120 minutes, the longest time point recorded. These results indicate that the IP GD approach offers a new method capable of generating steady state CBV maps.

The pulse sequence used to acquire images is another important factor. Both T₂ and T₂*-weighted images can be used in the gadolinium approach, each having its own advantages. T₂*-weighted images (GE) generate higher signal compared to T₂-weighted images (FSE). However, T₂*-weighted images (GE) have higher noise and can generate large artifacts particularly in air/bone regions (FIG. 3D). The ultimate trade-off on signal-to-noise remains unknown. T₂-weighted changes are more sensitive to microvasculature CBV and less sensitive to macrovasculature CBV (3, 21, 31). Since microvasculature CBV is more tightly coupled to neuronal metabolism, T₂-weighted changes provide a more accurate measure for the purposes of mapping brain dysfunction.

The route of contrast agent administration is a final factor that needs to be considered since it has practical implications. As discussed, IP administration is ideal for longitudinal analysis, and this route was taken based on the known anatomy of peritoneal microcirculation (32, 33), and its effect on peritoneal solute transport and ultrafiltration (17). Although it is clear that peritoneal solute transport is influenced by local hemodynamic changes induced by pH, buffers, etc, the exact dynamics of transport remain unknown (17). When IP gadolinium was administered at doses 50 times or higher than dose typically recommended for IV use in humans, a clear magnetic susceptibility perturbation was observed in the both in T₂ and T₂* weighted images after the initial 7.5 minutes, which reached a plateau phase after 37.5 minutes. It is worth noting that in contrast to studies of AR₂ after IV MION injections, which have found a linear relationship between the amounts of contrast agent administered and observed AR₂ (12), our experiments with IP GD injections (FIG. 1A) found no such linear relationship. This most likely reflects the complex dynamics of peritoneal solute transport. Since dynamics of peritoneal microcirculation is influenced by physiological variables such as cardiac output, vasoactive substances, and O₂ supply (17), these factors were controlled in part by monitoring different physiological parameters and used as criteria for image selection. It was also empirically established that using a IP flush at the end of each imaging session prevents residual GD from interfering with subsequent CBV mapping.

### REFERENCES

1. Kety, S S and Schmidt C F. The effects of altered arterial tensions of carbon dioxide and oxygen on cerebral blood flow and cerebral oxygen consumption of normal young men. J. Clin. Invest. 1948; 27:484-492.
2. Ogawa S, Lee T M, Barrere B. The sensitivity of magnetic resonance image signals of a rat brain to changes in the cerebral venous blood oxygenation. Magn. Reson. Med. 1993; 29(2): 205-10.
3. Boxerman J L, Hamberg L M, Rosen B R, Weisskoff R M. MR contrast due to intravascular magnetic susceptibility perturbations. Magn. Reson. Med. 1995; 34(4):555-66.
4. Frahm J, Bruhn H, Merboldt K D, Hanicke W. Dynamic MR imaging of human brain oxygenation during rest and photic stimulation. J. Magn. Reson. Imaging. 1992; 2(5):501-5.
5. Fox P T, Raichle M E. Stimulus rate dependence of regional cerebral blood flow in human striate cortex, demonstrated by positron emission tomography. J. Neurophysiol. 1984; 51(5):1109-20.
6. Edelman R R, Siewert B, Darby D G, Thangaraj V, Nobre A C, Mesulam M M, Warach S. Qualitative mapping of cerebral blood flow and functional localization with echo-planar MR imaging and signal targeting with alternating radio frequency. Radiology. 1994; 192(2):513-20.
7. Belliveau J W, Kennedy D N Jr, McKinstry R C, Buchbinder B R, Weisskoff R M, Cohen M S, Vevea J M, Brady T J, Rosen B R. Functional mapping of the human visual cortex by magnetic resonance imaging. Science. 1991; 254(5032):716-9.
8. Belliveau J W, Rosen B R, Kantor H L, Rzedzian R R, Kennedy D N, McKinstry R C, Vevea J M, Cohen M S, Pykett I L, Brady T J. Functional cerebral imaging by susceptibility-contrast. NMR. Magn. Reson. Med. 1990; 14(3):538-46.
9. Wu E X, Tang H, Asai T, Yan S D. Regional cerebral blood volume reduction in transgenic mutant APP (V717F, K670N/M671L) mice. Neurosci. Lett. 2004; 29; 365(3):223-7.
10. Lin W, Celik A, Paczynski R P. Regional cerebral blood volume: a comparison of the dynamic imaging and the steady state methods. J. Magn. Reson. Imaging. 1999; 9(1):44-52.
11. Small S A, Wu E X, Bartsch D, Perera G M, Lacefield C O, DeLaPaz R, Mayeux R, Stern Y, Kandel E R. Imaging physiologic dysfunction of individual hippocampal subregions in humans and genetically modified mice Neuron. 2000; 28(3):653-64.
12. Wu E X, Wong K K, Andrassy M, Tang H. High-resolution in vivo CBV mapping with MRI in wild-type mice, Magn. Reson. Med. 2003; 49(4):765-70.
13. Dunn J F, Roche M A, Springett R, Abajian M, Merlis J, Daghlian C P, Lu S Y, Makki M. Monitoring angiogenesis in brain using steady-state quantification of DeltaR2 with MION infusion. Magn. Reson. Med. 2004; 51(1):55-61.
14. Lin T N, Sun S W, Cheung W M, Li F, Chang C. Dynamic changes in cerebral blood flow and angiogenesis after transient focal cerebral ischemia in rats. Evaluation with serial magnetic resonance imaging. Stroke. 2002; 33(12):2985-91.
15. Okon E E, Pulikan D, Pereverzev A E, Kudriavtsev B N, Zhale P. Toxicity of magnetite-dextran particles: morphological study Tsitologiia. 2000; 42(4):358-66.
16. Filippi M, Rovaris M, Capra R, Gasperini C, Yousry T A, Sormani M P, Prandini F, Horsfield M A, Martinelli V, Bastianello S, Kuhne I, Pozzilli C, Comi G. A multi-centre longitudinal study comparing the sensitivity of monthly MRI after standard and triple dose gadolinium-DTPA for monitoring disease activity in multiple sclerosis. Implications for phase II clinical trials. Brain. 1998; 121 (Pt 10):2011-20.
17. Lameire N, Mortier S, De Vriese A. Peritoneal microcirculation. Contrib. Nephrol. 2003 (140):56-69.
18. Kuppusamy K, Lin W, Cizek G R, Haacke E M. In vivo regional cerebral blood volume: quantitative assessment with 3D T1-weighted pre- and postcontrast MR imaging. Radiology. 1996; 201(1):106-12.
19. Moreno H, Nava A, Tanji K, Puzzo D, Fukuyama H, Tang H, Westaway D, Arancio O, George Hyslop P, Small S. Imaging hippocampal dysfunction in transgenic mice with MRI. 9^{Th} International Conference on Alzheimer. Neurobiology of aging. 2004 (S 239) 17-22. Abstracts.
20. Lee S P, Silva A C, Ugurbil K, Kim S G. Diffusion-weighted spin-echo fMRI at 9.4 T: microvascular/tissue contribution to BOLD signal changes. Magn. Reson. Med. 1999; 42(5):919-28.
21. Weisskoff R M, Zuo C S, Boxerman J L, Rosen BR. Microscopic susceptibility variation and transverse relaxation: theory and experiment. Magn. Reson. Med. 1994; 31(6):601-10.
22. Ostergaard L, Smith D F, Vestergaard-Poulsen P, Hansen S B, Gee A D, Gjedde A, Gyldensted C Absolute cerebral blood flow and blood volume measured by magnetic resonance imaging bolus tracking: comparison with positron emission tomography values. J. Cereb. Blood Flow Metab. 1998; 18(4):425-32.
23. Hansen T D, Warner D S, Todd M M, Vust L J. The role of cerebral metabolism in determining the local cerebral blood flow effects of volatile anesthetics: evidence for persistent flow-metabolism coupling. J. Cereb. Blood Flow Metab 1989; 9(3):323-8.
24. Lei H, Grinberg O, Nwaigwe C I, Hou H G, Williams H, Swartz H M, Dunn J F. The effects of ketamine-xylazine anesthesia on cerebral blood flow and oxygenation observed using nuclear magnetic resonance perfusion imaging and electron paramagnetic resonance oximetry. Brain Res. 2001; 913(2): 174-9.
25. Paxinos G, Franklin K. The mouse brain in stereotaxic coordinates. Academic Press 2001.
26. Lev M H, Kulke S F, Sorensen A G, Boxerman J L, Brady T J, Rosen B R, Buchbinder B R, Weisskoff R M. Contrast-to-noise ratio in functional MRI of relative cerebral blood volume with sprodiamide injection. J. Magn. Reson 1997; 7(3):523-7.
27. van Zijl P C, Eleff S M, Ulatowski J A, Oja J M, Ulug A M, Traystman R J, Kauppinen R A. Quantitative assessment of blood flow, blood volume and blood oxygenation effects in functional magnetic resonance imaging. Nat. Med. 1998; 4(2): 159-67.
28. Reimer P, Tombach B. Hepatic MRI with SPIO: detection and characterization of focal liver lesions. Eur. Radiol. 1998; 8(7): 1198-204.
29. Oksendal A N, Hals P A. Biodistribution and toxicity of MR imaging contrast media. J. Magn. Reson. Imaging. 1993; 3(1): 157-65.
30. Tweedle M F, Wedeking P, Kumar K. Biodistribution of radiolabeled, formulated gadopentetate, gadoteridol, gadoterate, and gadodiamide in mice and rats. Invest. Radiol. 1995; 30(6):372-80.
31. Simonsen C Z, Ostergaard L, Smith D F, Vestergaard-Poulsen P, Gyldensted C. Comparison of gradient- and spin-echo imaging: CBF, CBV, and MTT measurements by bolus tracking. J. Magn. Reson. Imaging 2000; 12(3):411-6.
32. White R, Granger. The peritoneal microcirulation in peritoneal dialysis; in: Gokal R, Khanna R, Kreidiet R T, Nolph K D (eds): textbook of peritoneal dialysis. Dordrecht, Kluwer Academic Publishers 2000, pp 107-135.
33. Gotloib l, Shostak A, Wajsbrot V. Functional structure of the peritoneum as a dialyzing memebrane in: Gokal R, Khanna R, Kreidiet R T, Nolph K D (eds): textbook of peritoneal dialysis. Dordrecht, Kluwer Academic Publishers 2000, pp 37-106.

1. A method for determining the amount of blood in a volume of cerebral tissue (cerebral blood volume) in a mammalian subject comprising:
(a) acquiring a first magnetic resonance image of the volume of tissue in vivo;
(b) administering intraperitoneally to the subject a gadolinium-containing contrast agent in an amount greater than about 1 mg per kg body weight and less than about 20 mg per kg body weight;
(c) acquiring a second magnetic resonance image of the volume of tissue in vivo, which second image is acquired at least about 15 minutes after, but not more than about 2 hours after, administering the contrast agent; and
(d) determining the amount of cerebral blood volume based on the first and second images.

2-11. (canceled)

12. A method for determining the change in the amount of blood in a volume of cerebral tissue (cerebral blood volume) in a mammalian subject over a predefined period of time, comprising determining the cerebral blood volume at a plurality of time points during the predefined period of time and comparing the cerebral blood volumes so determined, so as to determine the change in the cerebral blood volume over the predefined period of time, wherein at each time point, determining the cerebral blood volume is performed according to the method of claim 1, with the proviso that at each time point other than the final time point in the predefined period of time, a saline solution is intraperitoneally administered to the subject following either step (c) or step (d).

13-31. (canceled)

32. A method for determining, in an animal model for a human neurological disease correlative with a decrease in cerebral blood volume, whether an agent inhibits the decrease, comprising:
(a) administering the agent to the subject;
(b) determining the cerebral blood volume of a suitable volume of the subject's cerebral tissue at a plurality of time points during a predefined period of time, wherein step (a) is performed before and/or during the predefined period; and wherein determining the cerebral blood volume is performed according to the method of claim 1, with the proviso that at each time point other than the final time point in the predefined period of time, a saline solution is intraperitoneally administered to the subject following either step (c) or step (d) of claim 1;
(c) comparing the cerebral blood volumes so determined, so as to determine the change in the cerebral blood volume over the predefined period of time; and
(d) comparing the change determined in step (c) with the change determined in a subject to which the agent was not administered, thereby determining whether the agent inhibits the decrease in cerebral blood volume correlative with the disease.

33-51. (canceled)
